(19) United States
(12) Patent Application Publication
(10) Pub. No.: US 2004/0265231 A1
(43) Pub. Date: Dec. 30, 2004

(54) Use of antioxidants to mitigate radioimmunotherapy-induced radiation toxicity

(75) Inventors: Rosalyn D. Blumenthal, West Orange, NJ (US); David M. Goldenberg, Mendham, NJ (US)

Correspondence Address: HELLER EHRMAN WHITE & MCAULIFFE LLP, 1666 K STREET,NW, SUITE 300, WASHINGTON, DC, 20006, US

(73) Assignee: Center for Molecular Medicine and Immunology, Belleville, NJ (US)

(21) Appl. No.: 10/893,358

(22) Filed: Jul. 19, 2004

Related U.S. Application Data
(63) Continuation of application No. 09/549,721, filed on Apr. 14, 2000, now abandoned.
(60) Provisional application No. 60/130,933, filed on Apr. 26, 1999.

(51) Int. Cl. A61K 51/00; A61K 31/355; A61K 31/07; A61K 31/375

(52) U.S. Cl. 424/1.49; 514/458; 514/474; 514/725

(57) ABSTRACT

The instant invention provides a method of using antioxidant, e.g., vitamins, as radioprotective agents to mitigate gastrointestinal and hemopoietic toxicity of the radioimmunotherapy., The instant invention further provides a method of combining the administration of antioxidant with BMT to produce an additive radioprotective effect against radiation damage to healthy tissues during the radioimmunotherapy.

### RELATED APPLICATIONS

This application claims priority from U.S. Provisional Application 06/103,933, filed Apr. 26, 1999.

### BACKGROUND OF THE INVENTION

Radiotherapy is an important form of tumor therapy. Various methods of radiotherapy have been developed to treat tumors. Among them, radioimmunotherapy (RAIT) has been applied broadly. It employs antibodies to direct radioisotopes to specific tissues and cells, thus enhancing specificity of tumor treatment and reducing toxicity. RAIT further reduces its side effects by using low dose rate radiation.

Radiation damage to healthy tissues and cells is a major problem associated with radiotherapy. Such damage has been primarily attributed to radiation-generated reactive oxygen species. Typical reactive oxygen species include the hydroxyl radical, superoxide anion radical, hydrogen peroxide, molecular oxygen, hypochlorite, the nitric oxide radical and peroxynitrite. These active oxygen species oxidize functionally important biological molecules, such as nucleic acids, carbohydrates, lipids and lipoproteins, and damage tissues and cells. They have been implicated in a variety of biological processes, e.g., antimicrobial defense, inflammation, carcinogenesis and aging. As reflected by body weight loss, myelosuppression and blood cell loss, such as decreased white blood cell (WBC) and platelet counts, gastrointestinal and hematopoietic toxicity are the most notable consequences of the radiation damage. The toxicity severely limits the radiation dosage of RAIT and reduces the effectiveness of tumor treatment.

A number of methods have been developed to mitigate the hematopoietic toxicity of radiation. Stem cell transplantation (SCT) and bone marrow transplantation (BMT) are the most frequently used methods. Other methods include using cytokines to stimulate the immune system and hemoregulatory proteins such as HP5b to turn off hematopoiesis during the radiation exposure period. These methods have achieved various degrees of success in combating hematopoietic toxicity. The gastrointestinal toxicity, however, has never been dealt with directly.

Because the radiation damage is attributable to active oxygen species, antioxidants become rational candidates for mitigation. Antioxidant vitamins, such as vitamins A, C and E, have been reported to reduce DNA damage, diminish lipid peroxidation and increase tissue radioresistance (Sies, H. and Stahl, W., Vitamins E and C. β-carotene, and other carotenoids as antioxidants, 62 Am. J. Clin. Nutr. 1315S (1995)). One murine study reported that both vitamins C and E exhibited radioprotective effects as illustrated by a reduced frequency of micronuclei and chromosomal aberration post-radiation (Sarma, L. and Kesavan, P. C., Protective effects of vitamins C and E against gamma-ray-induced chromosomal damage in mouse, 63 (6) Int. J. Radiat. Biol., 759 (1993)). Other studies, however, have reported that vitamin E alone was ineffective in rats and mice (el-Nahas, S. M. et al., Radioprotective effects of vitamins C and E, 301(2) Mutat. Res. 143 (1993); Umegaki, K. et al., Effect of vitamin E on chromosomal damage in bone marrow cells of mice having received low dose of X-ray irradiation, 64(4) Int. J. Vitam. Nutr. Res. 249 (1994)).

Limited work has been done on the efficacy of antioxidant vitamins as radioprotectors against tissue incorporated radionuclides. One study has reported that both dietary and injected vitamin C exhibited radioprotective effects against internalized ¹³¹I and ¹²⁵I, but not against alpha emission from ²¹⁰Po (Narra, V. R. et al., Vitamin C as a radioprotector against iodine-131 in vivo, 34 J. Nucl. Med. 637 (1993)). Another study has reported that vitamin A is an effective radioprotector against tissue incorporated I5I, but not against ²¹⁰Po (Harapanhalli, R. S. et al., Vitamins as radioprotectors in vivo II. Protection by vitamin A and soybean oil against radiation damage caused by internal radionuclides, 139 Radiat. Res. 115 (1994)).

Antioxidants or antioxidant vitamins have never been used to mitigate the side effects of RAIT. It could not be predicted whether or not antioxidants may protect the tumor tissues to be treated, as well as normal tissues, and thus reduce the effectiveness of RAIT. A need therefore continues to exist for methods of mitigating the radiation side effect of RAIT.

### SUMMARY OF THE INVENTION

One object of the present invention is to provide a method for mitigating the radiation side effects of RAIT, particularly the hematopoietic and gastrointestinal toxicity, with antioxidants.

Another object of the present invention is to achieve synergistic or additive effects in reducing RAIT-induced gastrointestinal and hemotopoietic toxicity by applying multiple antioxidant vitamins.

Another object of the present invention is to achieve synergistic or additive effects of radioprotection by combining antioxidant vitamins with BMT.

Yet another object of the present invention is to determine the proper dose and route of administration for the antioxidant vitamins to achieve the most desirable radioprotection against tissue damage by RAIT.

In accomplishing these and other objects of the invention, there is provided, in accordance with one aspect of the present inventions, a method for mitigating the side effects of RAIT comprising administering a targeted cytotoxic radioisotope to a disease site, wherein the improvement comprises mitigating the radiation toxicity by administering at least one antioxidant, which, includes but is not limited to, antioxidant vitamins such as vitamins A, C and E. In another embodiment, a combination of two or more antioxidant vitamins selected from the group consisting of vitamins A, C and E is administered. In a preferred embodiment, a combination of vitamins A, C and E is administered.

In accordance with another aspect of,the present invention, at least one of the antioxidant vitamins is administered at a dosage 5 to 10 fold over its regular dosage as a vitamin, and preferably each is so administered. In a preferred embodiment, the antioxidant vitamins are administered several days before the application of the radioisotope. In another preferred embodiment, the radioimmunotherapy is administered in combination with a treatment selected from the group consisting of:

bone marrow transplantation,

stem cell transplantation,

administration of hemoregulatory peptide, and

administration of an immunomodulation agent.

Additional objects and advantages of the invention are set forth in part in the description that follows, and in part will be obvious from the description, or may be learned by practice of the invention.

### BRIEF DESCRIPTION OF THE DRAWINGS

FIG. 1 is a plot of the peripheral white blood cell counts on days 7, 14, and 21, post either a 400 or 500 μCi dose of ¹³¹I-MN-14 IgG. Mice were either left untreated, or given BMT, vitamins, or both vitamins and BMT. The average of five (5) mice is recorded.

FIG. 2 shows the platelets measured on day 14. The mean of five (5) mice in each treatment group is recorded.

FIG. 3 summarizes the results of the study comparing the RAIT efficacy with or without administration of radioprotective vitamins.

### DETAILED DESCRIPTION OF THE INVENTION

The present invention describes a method of mitigating the toxicity of radioimmunotherapy (RAIT). Generally, RAIT employs an antibody conjugated with a radioisotope such as ¹³¹I. The antibody binds specifically to targeted tumor tissue, thus bringing radiation close to the targeted tumor tissue. The radiation kills the tumor tissue, but also damages some healthy tissues. In one embodiment of the invention, the method comprises the administration of an antibody targeting cytotoxic radioisotope to a disease site and the improvement comprises the administration of an antioxidant, which protects the healthy tissues from the radiation.

In an aerobic organism, a delicate balance of oxidants and anti-oxidants maintains a steady physiological environment. The radiation of RAIT generates excess oxidants which shift the balance and lead to cell and tissue damage. Antioxidants afford an important defense mechanism against the excess oxidants. An antioxidant is defined as a substance that reduces oxidation of a substrate such as DNA and lipid. It can inhibit the oxidation at a low concentration compared to that of the substrate. There are two groups of antioxidants: (1) hydrophilic antioxidants, such as ascorbate, glutathione and selenium, and (2) lipophilic antioxidants, such as tocopherols, carotenoids, carotenes and lycopene. These antioxidants are often observable in blood plasma. Antioxidants have been associated with lowered DNA damage, diminished lipid peroxidation or inhibited malignant transformation in vitro.

In particular, an antioxidant vitamin, such as vitamin A, C or E, is administered in conjunction with RAIT. Antioxidant vitamins are attractive candidates for mitigating RAIT toxicity because they are readily available and generally inexpensive. Their toxicity, such as mutagenicity and carcinogenicity, is low even ingesting large amounts.

Vitamin C, often synonymously referred to as ascorbic acid, L-ascorbic acid and ascorbate, is the major hydrophilic antioxidant. It is considered to be the most important antioxidant in extracellullar fluids. Under most physiological conditions, vitamin C exhibits many cellular activities of an antioxidative nature. In aqueous phase, vitamin C efficiently scavenges various free radicals, such as hydroxyl radical and peroxyl generated by superoxide, hydrogen peroxide and hypochlorite, and protects bio-membranes from peroxidative damage. In studies with human plasma lipids, vitamin C exhibited far more effective inhibitory effects on radical initiated lipid peroxidation than other antioxidants such as protein thiols, urate, bilirubin and α-tocopherol. Frei B. et al., Ascorbate is an outstanding antioxidant in human blood plasma, 86 Proc. Natl. Acad. Sci. USA 6377 (1989). In addition, vitamin C has also been reported to protect against endogenous oxidative DNA damage in human sperm.

Vitamin E is the most abundant lipophilic antioxidant. It embraces a group of compounds including tocopherols, tocopherol homologs and tocotrienols. In humans, the biologically and chemically most active form of vitamin E is α-tocopherol, which presents in biologic membranes and lipoproteins. Alpha-tocopherol effectively breaks the free radical chain reaction and inhibits lipid peroxidation.

Vitamin A is a member of the carotenoids family, which encompasses more than 500 lipophilic natural compounds. Beta-carotene, the most important member of the family, is the precursor of vitamin A. For the claims of this patent application, β-carotene and vitamin A are used interchangeably. Beta-carotene and other carotenoids such as lycopene exert their antioxidant function through physical quenching of molecular oxygen and other electronically excited molecules. Most carotenoids contain extended conjugated double bonds, responsible for the antioxidant activity such as inhibiting free radical reactions. At a low concentration and a partial pressure similar to those found in most tissues under physiologic conditions, β-carotene can inhibit the oxidation of model compounds, suggesting its capacity to protect tissues against oxidative damage under normal physiological conditions. In spite of individual differences in tissue distribution of carotenoids, liver, adrenal gland and testes have always been found to contain significantly more β-carotene, implicating a varied degree of protection to different tissues. Compared with α-tocopherol, β-carotene is a relatively weak antioxidant.

In accordance with another aspect of the present invention, the improvement of RAIT comprises the administration of a combination of two or more antioxidants selected from the group consisting of antioxidant vitamins A, C and E. In a preferred embodiment, a vitamin mix of vitamins A, C and E is administered to achieve the maximum radioprotective effect. Due to difference in hydrophilicity, vitamins A, C and E have different subcellular distributions and consequently protect against different forms of free radical damages by RAIT. The hydrophilic vitamin C presents in large quantity in extracellular matrix and scavenges free radicals in aqueous phase effectively. The lipophilic vitamin E presents in biomembranes and protects the membranes from peroxidation. Vitamin A is more lipophilic than vitamin E. It likely presents at the interior of membranes and scavenges radicals more efficiently than vitamin E within lipophilic compartment. Niki E. et al., Interaction among vitamin C. vitamin E, and beta-carotene, 62 Am. J. Clin. Nutr. 1322S (1995). The interactions of vitamins A, C and E further favor the application of their combination for improving RAIT toxicity. In vitro studies have revealed that vitamins A and E synergistically inhibit lipid peroxidation. The synergy is partly attributed to the facts that vitamins A and E protect each other against consumption. Tesoriere, L. et al., Synergistic interaction between vitamin A and vitamin E against lipid peroxidation in phosphatidylcholin liposomes, 32 Atch Biochem. Biophys. 57 (1996). Both β-carotene and vitamin C have been reported to significantly enhance the circulating concentration of vitamin E (14). Vitamin C can also restore the radical scavenging activity of tocopherol as suggested by in vitro studies. Stoyanovsky, D. et al., Endogenous ascorbate regenerates vitamin E in the retina directly and in combination with dihydrolipoic acid, 14 Curr. Eye. Res. 181 (1995).

In another embodiment, the antioxidants are administered prior to RAIT treatment. In a preferred embodiment, the antioxidants are administered several days (e.g., three days) before RAIT treatment, allowing vitamins, particularly vitamins A and E, to be stored up. The half-life of the active oxygen species generated by radiation varies from nanoseconds to seconds. Damage by these active oxygen species would be expected to result shortly after their generation. It is therefore effective to place the antioxidants in a position to intercept the active oxygen species prior to their generation. Discrepancies in reports regarding the radioprotective effects of the antioxidants possibly result from the difference in the time of administration in relation to the radiation treatment. For example, vitamins administered two hours before or immediately after the radiation produced the greatest protective effect, but no protection when administered two hours afterwards. Sarma, L. and Kesavan, P. C., Protective effect of vitamins C and E against gamma-ray-induced chromosomal damage in mouse, 63 Int. J. Radiat. Bio. 759 (1993).

In accordance with another aspect of the present invention, a preferred embodiment of the invention comprises administration of a much higher vitamin dosage than that used as ordinary vitamins. Generally, for humans, vitamin A dosage ranges from 25,000 to 50,000 IU (international units) per day, vitamin E dosage ranges 150 to 300 IU per day, and vitamin C dosage ranges from 1,500 to 3,000 mg per day. For the route of administration, vitamins are usually given orally pre-RAIT. Nonetheless, because RAIT often damages gastrointestinal mucosa and prevents maximum absorption through oral administration, intravenous (i.v.) or intromuscular (i.m.) administration is generally preferred for post-RAIT treatment

The present invention further discloses a method of combining the antioxidant treatment with other means for mitigating RAIT toxicity, such as BMT, SCT and administration of hemoregulatory peptide or immunomodulation agents. In a preferred embodiment, a method of mitigating RAIT toxicity comprises BMT and administration of antioxidant vitamins. In the most preferred embodiment, the mix of vitamins A, C and E are administered in conjunction with BMT to mitigate RAIT toxicity. Bone marrow (BM) is collected from the patient, who does not have tumor metastatic sites growing in bone, or from a matched donor and stored frozen with cryopreservatives. At about 5-14 days, usually 7 days, after RAIT, the stored BM is thawed. After washing, assessing cell viability and counting the cell, the BM cells in amount of 10⁷ to 10⁸ are reinfised intravenously. Generally, the vitamins are administered before RAIT and are continuously administered at least 11 days post-RAIT.

A risk of using radioprotective antioxidant vitamins to reduce RAIT toxicity is that the vitamins may compromise the therapeutic efficacy of RAIT if they protect the healthy and tumor tissues indiscriminately. Experiments have been carried out to evaluate the impact of vitamin administration on RAIT efficacy of halting tumor growth. No adverse effects on RAIT efficacy were observed. Therefore, the administration of antioxidant vitamins reduces the dose-limiting side effects of RAIT and permits radioantibody dose intensification without compromising the therapeutic benefit.

### EXAMPLES

The embodiments of the invention are further illustrated through the following examples which show aspects of the invention in detail. The examples illustrate specific elements of the invention and are not to be construed as limiting the scope thereof.

### Example 1

### Vitamin Administration Increases Mice MTD for RAIT

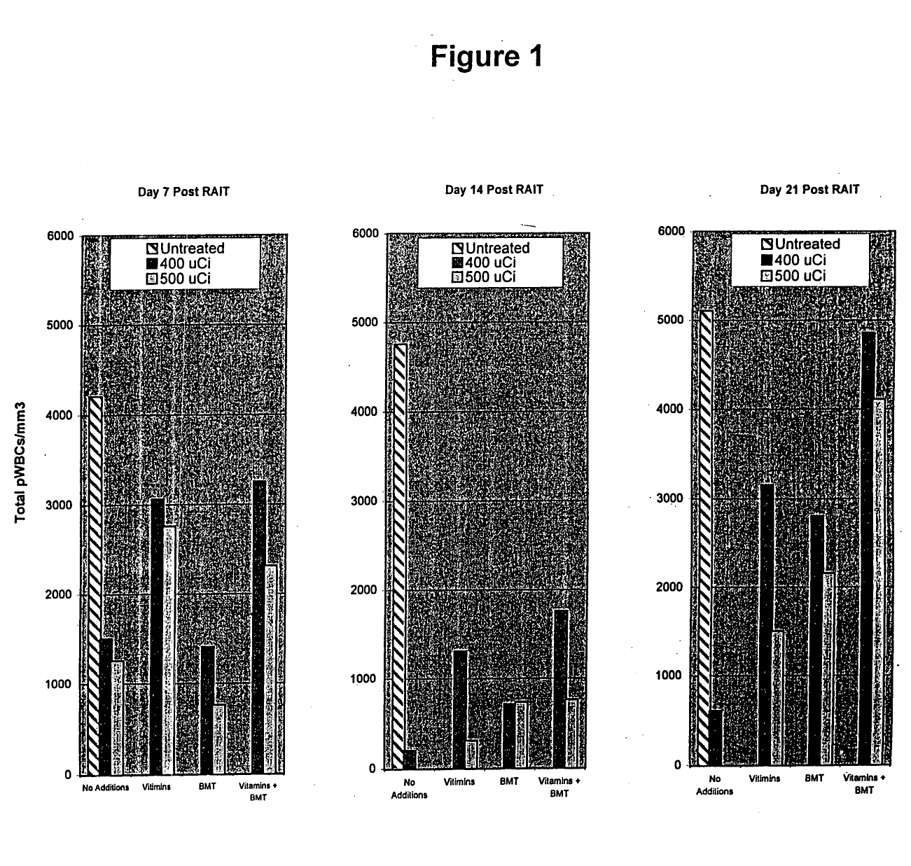

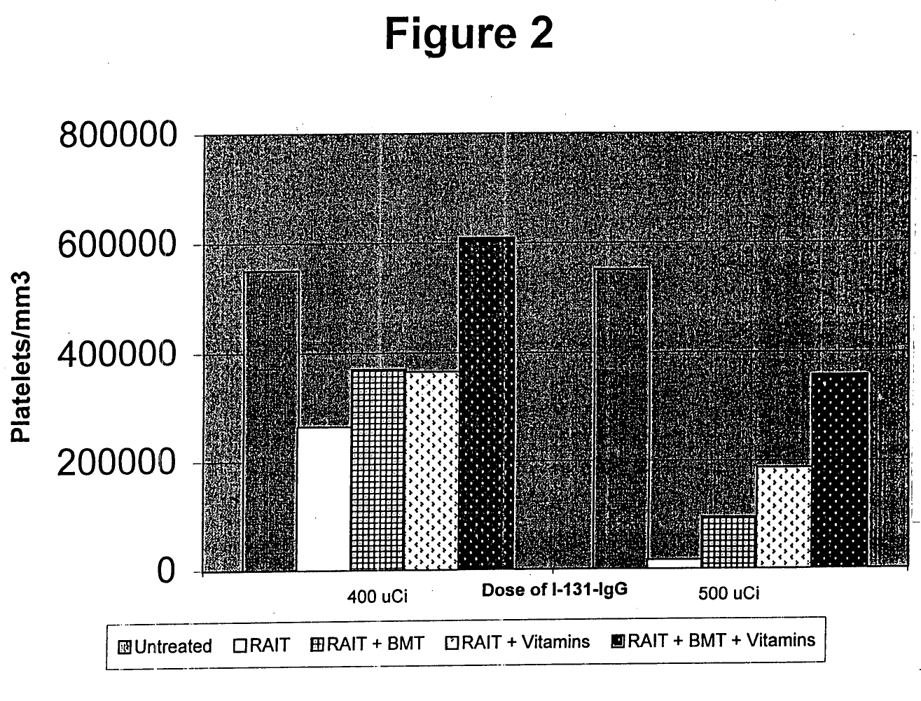

Six days before RAIT, a mixture of vitamins A, C and E was administered to the experimental non-tumor bearing nude mice through a water bottle containing 2 grams per liter of the vitamin mix, which equals to a concentration of 1,400 IU vitamin A, 7 IU vitamin E and 45.5 mg vitamin C per liter. On a daily basis, a mouse was given 40 IU of vitamin A, 0.2 IU of vitamin E and 1.3 mg of vitamin C by such a delivery method. Starting at the day of RAIT, the vitamins were infused into the mice through an implanted 14-day osmotic pump because RAIT decreases water intake of the mice. The pump delivered to a mouse the equivalent of 21.3 IU/d vitamin A, 0.11 IU/d vitamin E and 0.47 mg/d vitamin C. Over a fourteen-day period, 225 μl of the vitamin mix containing 298 IU of vitamin A, 1.54 IU of vitamin E and 6.58 mg of vitamin C were delivered to a mouse. The radioantibody ¹³¹I-MN-14 IgG was used for RAIT. The starting dose was 350 μCi, the maximal-tolerated dose (MTD) in non-tumor bearing mice for ¹³¹I-MN-14 IgG. The dose was then escalated up to 500 μCi. Tables 1 and 2 and FIGS. 1 and 2 present the survival rate, the body weight and peripheral white blood cell and platelet count of the experimental mice at different times after RAIT.

TABLE 1
Survival of Nude Mice Given a Single Dose of RAIT (¹³¹I-MN-14 IgG)

| | | +Vitamin | | +BMT & |
|---|---|---|---|---|
| Dosage | RAIT Alone | Mix | +BMT* | Vitamin Mix |
| 350 μCi | 100% | N/A | N/A | N/A |
| 400 μCi | 20% | 70% | 100% | 100% |
| 450 μCi | N/A | 50% | 100% | N/A |
| 500 μCi | 0% | 20% | 70% | 100% |

*107 donor BM cells infused i.v. on day 7.

Although the amount of vitamins delivered was not optimized, for example, vitamins E and C were well below the optimal amount, the vitamins raised the survival rate of nude mice from 20% to 70% when 400 μCi of ¹³¹ I-MN-14 IgG was used. At a dose of 500 μCi, the vitamin mix increased the survival rate from zero to 20%.

Combining the vitamins with BMT achieved an apparent additive enhancement of survival rate. At a dose of 500 μCi of ¹³¹I-MN-14 IgG, the vitamin mix and BMT increased the survival rate of nude mice 20% and 70%, respectively, while combining the vitamin mix with BMT, the mice survived 100%.

The data in Table 1 suggest a 150 μCi increase in MTD when a combination of the vitamin mix and BMT is used to mitigate the RAIT toxicity. Upon optimizing the vitamin dose, MTD should further increase. A 150 μCi dose increase in mice will likely translate into a much higher dose increase in patients, as has been shown for BMT/SCT in mice and humans. For example, while BMT permits a 30% dose increase in mice, a similiar treatment would permit a 300-400% dose increase in humans. Since the applicable RAIT dosage for human generally ranges from 60 to 70 mCi, the administration of vitamins and BMT could increase this applicable dosage to as high as 180-280 mCi. Such an increase would predictably also increase the efficacy of RAIT.

TABLE 2
Percent Change in Body Weight Post-RAIT

| | | | | | +BMT & |
|---|---|---|---|---|---|
| Dosage | Control | RAIT Alone | +BMT | +Vitamin Mix | Vitamin Mix |
| 400 μCi | +10.2 ± 1.9 | −5.5 ± 2.4 | −7.4 ± 0.7 | +0.6 ± 0.3 | +0.8 ± 2.4 |
| (day 7) | | | | (p < 0.001) | (p < 0.001) |
| 500 μCi | | −10.0 ± 2.0 | −10.2 ± 1.3 | −1.8 ± 1.3 | −1.3 ± 4.7 |
| (day 7) | | | | (p < 0.001) | (p < 0.01) |
| 400 μCi | +18.1 ± 1.4 | −0.4 ± 1.6 | −2.8 ± 1.5 | +12.5 ± 8.2 | +9.1 ± 1.9 |
| (day 14) | | | | (p < 0.001) | (p < 0.001) |
| 500 μCi | | −20.7 ± 4.1 | −19.9 ± 8.2 | −1.4 ± 2.8 | +2.7 ± 0.9 |
| (day 14) | | | | (p < 0.001) | (p < 0.001) |

The administration of the vitamin mix produced a clear protective effect against gastrointestinal toxicity as measured by decreased body weight loss. Table 2 shows weight loss data recorded as a percent of the total body weight on day 7 and 14 after either a 400 μCi or 500 μCi RAIT. Without the vitamins, the two doses result in 5.5% and 10% weight loss at day 7 after RAIT and 0.4% and 20.7% weight loss at day 14 after RAIT. For the same two RAIT doses, mice given the vitamin mix exhibited a 0.6% weight gain and a 1.8% weight loss at day 7 and a 12.5% weight gain and a 1.4% weight loss at day 14.

As shown by FIGS. 1 and 2, administration of the vitamins reduced the magnitude of RAIT-induced myelosuppression. FIG. 1 illustrates the effect of the vitamin mix, BMT, and the combination of the vitamin mix and BMT on peripheral WBC counts following a 400 μCi and 500 μCi RAIT treatment. As early as day 7 after RAIT, the vitamin mix increased WBC counts from 1464±418/mm³ to 3023±987/mm³ (p<0.02) following the 400 μCi RAIT and from 1235±705/mm³ to 2673±638/mm³ (p<0.01) following the 500 μCi RAIT.

On day 14 post a 400 μCi RAIT, BMT and the vitamin mix had an apparent additive effect on mice WBC counts. WBC counts in mice that were treated with neither the vitamin mix nor BMT were 154±43/mm³. WBC counts in mice treated with either BMT or the vitamin mix were 588±203/mm³ (p<0.01) and 1259±148/mm³, respectively. WBC counts in mice treated with both BMT and the vitamin mix, however, reach 1734±588/mm³ (p<0.001 compared with those given only BMT). Similar additive effects were also noted for 21 days post-RAIT. In these experiments, the vitamin mix was given to the mice several days before the RAIT, and BMT was given to the mice 7 days after RAIT. FIG. 2 demonstrates that the vitamin mix protects the platelet population, and that the administration of the vitamin mix in conjunction with BMT has additive protective effects on the platelet population.

### Example 2

### Vitamin Administration does not Adversely Affect RAIT Efficacy

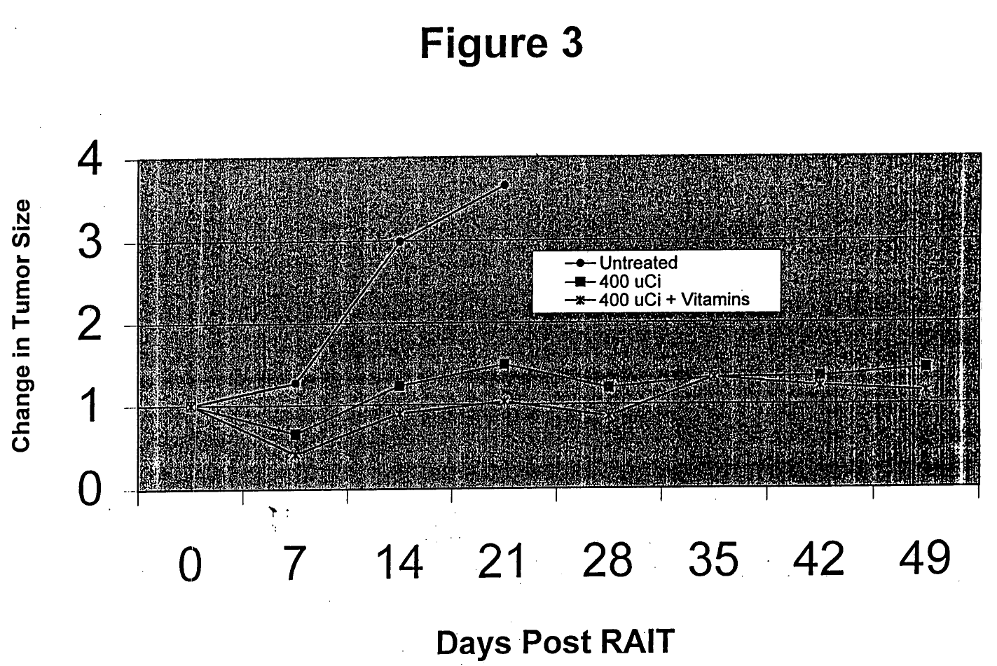

The GW-39 tumor-bearing nude mouse model was used to evaluate the impact of the vitamin administration on the efficacy of RAIT in halting tumor growth. FIG. 3 summarizes the results. For control mice which were not treated with RAIT, the tumor size increased 3.66±0.67 fold over a three-week period. For mice treated with a dose of 300 μCi ¹³¹I-MN-14 IgG alone, the tumor size only increased 1.2-1.5 fold over a similar period of time, between day 14 to day 49 post-RAIT. For mice treated with the same dose of RAIT and a dose of vitamins similar to the dose schedule used in example 1, the tumor size increased 0.9 to 1.3 fold during the same period. There was no significant difference in the pattern of tumor growth between the two groups of RAIT-treated mice. With or without vitamin administration, the RAIT treatment significantly slows down the tumor growth on these tumor-bearing nude mice.

It will be appreciated that other embodiments of the invention will be readily apparent to those of skill in this art and such variants are intended to be embraced by the scope of the appended claims.

What is claimed is:

1. In a method of radioimmunotherapy, wherein an antibody linked with cytotoxic radioisotope is administered to a disease site, the improvement comprising mitigating the radiation toxicity by administering at least one antioxidant.

2. A method according to claim 1, wherein said antioxidant is an antioxidant vitamin.

3. A method according to claim 2, wherein said vitamin is vitamin A.

4. A method according to claim 2, wherein said vitamin is vitamin C.

5. A method according to claim 2, wherein said vitamin is vitamin E.

6. A method according to claim 2, wherein a combination of two or more vitamins selected from the group consisting of vitamins A, C and E is administered.

7. A method according to claim 2, wherein a combination of vitamins A, C and E is administered.

8. A method according to claim 2, wherein at least one of said vitamins is administered at a dosage 5 to 10 fold over its regular dosage as a vitamin.

9. A method according to claim 2, wherein said vitamin is administered several days before radioimmunotherapy.

10. A method according to claim 1, wherein said radioimmunotherapy is administered in combination with a treatment selected from the group consisting of:
bone marrow transplantation,
stem cell transplantation,
administration of a hemoregulatooy peptide, and
administration of an immunomodulation agent.

11. A method according to claim 9, wherein at least one of said vitamins is administered at a dosage 5 to 10 fold over its regular dosage as a vitamin.

12. A method according to claim 10, wherein at least of said vitamins is administered at a dosage 5 to 10 fold over its regular dosage as a vitamin.

13. A method according to claim 10, wherein said at least one vitamin is administered several days before radioimmunotherapy.

14. A method according to claim 13, wherein at least one of said vitamins is administered at a dosage 5 to 10 fold over its regular dosage as a vitamin.
